(19) United States
(12) Patent Application Publication
(10) Pub. No.: US 2025/0213883 A1
(43) Pub. Date: Jul. 3, 2025

(54) PHOTODYNAMIC THERAPY DEVICES, SYSTEMS, AND METHODS

(71) Applicant: The United States Government as represented by the Department of Veterans Affairs, Washington, DC (US)

(72) Inventors: Jorge Antonio T. Gutierrez, Durham, NC (US); Sunil V. Rao, Durham, NC (US)

(21) Appl. No.: 18/867,225

(22) PCT Filed: May 26, 2023
(86) PCT No.: PCT/US2023/023663

§ 371 Date: Nov. 19, 2024

Related U.S. Application Data
(60) Provisional application No. 63/346,509, filed on May 27, 2022.

(51) Int. Cl. A61N 5/06; A61M 25/10; A61N 5/067

(52) U.S. Cl. CPC A61N 5/0601; A61N 5/062; A61N 5/067; A61M 25/10; A61N 2005/0602; A61N 2005/063; A61N 2005/0651; A61N 2005/0662

(57) ABSTRACT

A system for providing photodynamic therapy includes an assembly that is at least partially insertable into a patient. The assembly includes a catheter having an insertional end portion. The catheter is configured to permit positioning of the insertional end portion at a treatment site of an artery having an interior wall surface. At least one light emitting structure is coupled to the insertional end portion of the catheter. The assembly is configured to position the at least one light emitting structure at or against the interior wall surface of the artery.

### CROSS-REFERENCE TO RELATED APPLICATION

This application claims priority to and the benefit of the filing date of U.S. Provisional Patent Application No. 63/346,509, filed May 27, 2022, the entire contents of which are incorporated by reference herein for all purposes.

### FIELD

The application is generally related to systems and methods for providing photodynamic therapy to arteries to treat inflammation.

### BACKGROUND

Currently, there are few therapies to effectively treat atherosclerotic disease that accumulates in the arteries below the knee (anterior tibial, posterior tibial, and peroneal arteries). The most common therapies include balloon angioplasty and atherectomy. These therapies may provide a short-term solution to arterial blockages, but do little to mitigate the re-accumulation of atherosclerosis in these vessels.

### SUMMARY

Disclosed herein, in one aspect, is a system including an assembly that is at least partially insertable into a patient. The assembly includes a catheter having an insertional end portion. The catheter is configured to permit positioning of the insertional end portion at a treatment site of an artery having an interior wall surface. At least one light emitting structure is coupled to the insertional end portion of the catheter. The assembly is configured to position the at least one light emitting structure at or against the interior wall surface of the artery.

In one aspect, a method includes delivering at least one photosensitizer to a patient. At least one light emitting structure of an assembly is positioned within an artery of the patient. The assembly includes a catheter having an insertional end portion and the at least one light emitting structure. The at least one light emitting structure is coupled to the insertional end portion of the catheter. Light is delivered to an interior wall surface of the artery by the at least one light emitting structure of the assembly,

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a schematic diagram of a system for providing photodynamic therapy as disclosed herein.

FIG. 2A is a cross-sectional view of an assembly as disclosed herein in a deflated configuration. FIG. 2B is a cross-sectional view of the assembly of FIG. 2A in an inflated configuration.

FIG. 3 is a partial schematic side view of an assembly as disclosed herein.

FIG. 4 is a schematic view of an optical fiber for delivering light to a treatment site as disclosed herein.

FIG. 5 is a perspective view of a portion of a catheter having a resiliently elastic helical portion as disclosed herein.

FIG. 6 is a schematic view of the optical fiber of FIG. 5 in a sleeve for insertion into a patient as disclosed herein.

FIG. 7 shows a schematic diagram of a light dispersion structure as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein: rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15 degrees, up to 10 degrees, up to 5 degrees, or up to one degree (above or below) of the particularly stated angular value can be included within the scope of those aspects.

The word “or” as used herein means any one member of a particular list and, unless context dictates otherwise, in other aspects, can also include any combination of members of that list.

In the following description and claims, wherever the word “comprise” or “include” is used, it is understood that the words “comprise” and “include” can optionally be replaced with the words “consists essentially of” or “consists of”' to form another embodiment.

As used herein, “insertional” can be understood to refer to an end of a structure that is positioned away from or in a direction away from the clinician or clinician's control device.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow: plain meaning derived from grammatical organization or punctuation: and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

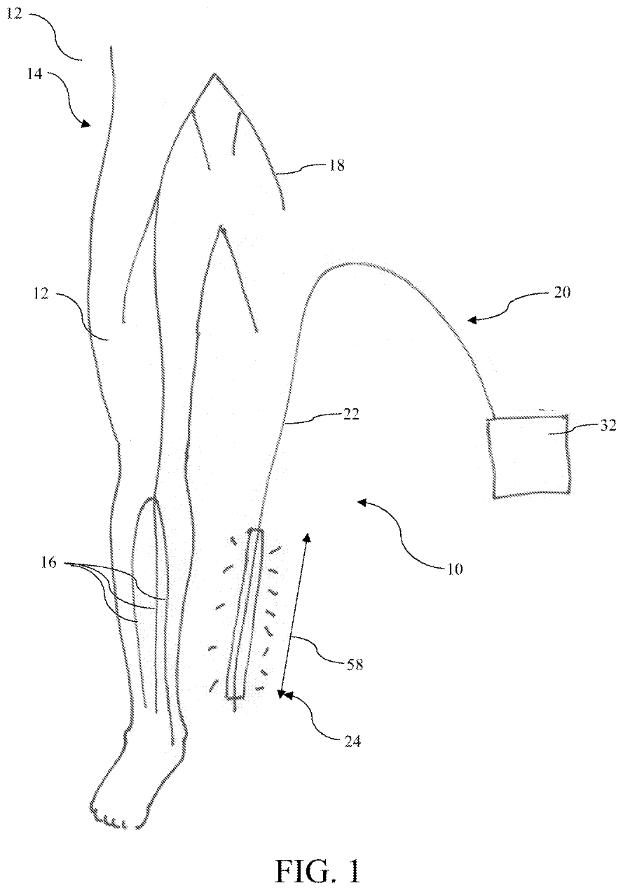

Disclosed herein, and with reference to FIGS. 1 and 3, is a system 10 for providing photodynamic therapy. The system 10 can be configured to provide photodynamic therapy to an artery, such as, for example, an artery in a leg 12 of a patient 14. In some aspects. the arteries can be tibial arteries 16. The arteries can have an interior wall surface 17. In some aspects, the treated tibial artery can be accessed from a femoral artery 18 (e.g., a contralateral femoral artery) or from an ipsilateral tibial artery 19.

The system 10 can comprise an assembly 20 that is at least partially insertable into the patient 14. The assembly 20 can comprise a catheter 22 having an insertional end portion 24. As used herein, the term “catheter” refers generally to a structure comprising medical grade materials and is configured for insertion into a patient. Optionally, the catheter 22 can have a flexible, tubular structure and can define a conduit or lumen. The catheter 22 can be configured to permit positioning of the insertional end portion 24 at a treatment site of the artery. For example, catheter 22 can have a sufficient working length (e.g., from about 90 cm to about 120 cm.) to extend to the artery from the contralateral femoral artery 18.

At least one light emitting structure 30 can be coupled to the insertional end portion 24 of the catheter 22. The assembly 20 can be configured to position the at least one light emitting structure 30 at or against the interior wall surface 17 of the artery 16.

In some optional aspects. the at least one light emitting structure 30 can be configured to emit light generated from an external light source 32 that is outside of the body of the patient 14. For example, in some aspects, the assembly 20 can further comprise at least one optical fiber 34 having an insertional first end 36 portion and an opposed second end portion 38. The optical fiber 34 can comprise a core 40 and a cladding 42 circumferentially enclosing at least a portion of the core.

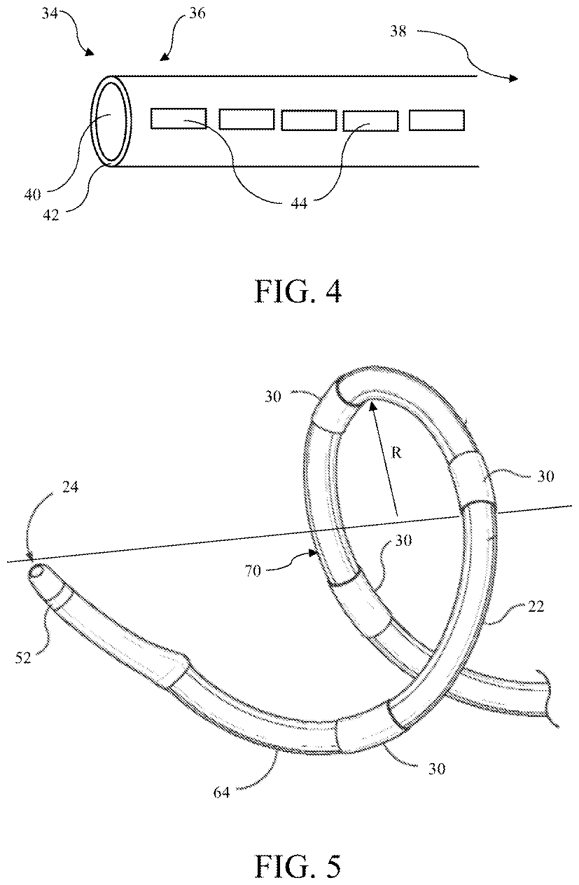

Referring to FIG. 4, the light source 32 (FIG. 1) can be configured to deliver light into the second end portion 38 of the optical fiber 34. The light source 32 that is outside the body can be a laser, such as, for example, a diode laser, an argon laser, a dye laser, a solid state laser such as Nd:YAG, or any other suitable light source capable of producing light at a suitable wavelength. In some optional aspects, the light source 32 can be configured to emit light at a wavelength from about 300 nm to about 600 nm. It is further contemplated that any wavelength can be selected based on the particular treatment.

In some aspects, the at least one light emitting structure 30 can comprise a portion of the cladding 42 that is configured to permit light to pass therethrough. For example, the portion of the cladding 42 that is configured to permit light to pass therethrough can comprise at least one aperture 44. In some optional aspects. the at least one aperture 44 can comprise a plurality of apertures that are axially spaced along the optical fiber 34. In further aspects, the aperture 44 can be a single aperture. Optionally, the single aperture can extend circumferentially around the core 40 to permit 360 degree radial emission. In some aspects, the at least one aperture 44 can be axially elongate along the length of the optical fiber 34.

In further aspects, a light dispersion structure 35 (FIG. 7) can couple to the insertional end portion of the core 40 of the optical fiber. The light dispersion structure can comprise reflecting structures, refracting structures, diffracting structures. or some combination thereof to direct light radially outwardly and against the interior wall surface of the artery 16.

In further aspects, the at least one light emitting structure 30 can comprise one or more light emitting devices 50. Thus, the light for treatment can be provided by a light source that is inserted into the patient. The light emitting device(s) 50 can be one or more LEDs, one or more super luminescent diodes, one or more laser diodes, one or more light emitting polymers, or combinations thereof. Optionally, the light emitting device(s) can comprise one or more organic LEDs. In some optional aspects, the light emitting devices 50 can be configured to emit light at a wavelength from about 300 nm to about 600 nm.

In some aspects, the assembly 20 can comprise a marker 52 at a known position relative to the at least one light emitting structure. The marker 52 can be opaque to a particular emission in order to be used for angiographic guidance.

The catheter 22 can have a length. In some aspects, assembly 20 can comprise a plurality of light emitting structures 30 that are spaced along the length of the catheter 22. For example, the assembly 20 can comprise a plurality of light emitting device(s) 50. In further aspects, the assembly 20 can comprise a plurality of apertures 44 that are spaced along the length of the catheter. In some aspects, the light emitting structure(s) 30 can be elongate along the length of the catheter.

Referring to FIG. 3, in some optional aspects, the at least one light emitting structure 20 can be configured to emit light along a treatment length 58 from about 50 mm to about 200 mm. For example, the assembly 20 can comprise a plurality of light emitting devices 50 along the axis of the catheter 22, wherein axially opposed light emitting devices 50 of the plurality of light emitting devices are spaced by from about 50 mm to about 200 mm. In further aspects, a single light emitting device 50 can extend along the length catheter from about 50 mm to about 200 mm.

In some aspects, the system 10 can comprise a guidewire 54. The catheter 22 can define an inner lumen 56 that is configured to receive the guidewire 54 therein.

The guidewire can be sufficiently rigid to position the insertional end portion 24 of the catheter 22 and the at least one light emitting structure 30 in an artery below a knee of the patient. For example, in some aspects, the guidewire can have a diameter from about 0.010 inches to about 0.05 inches (e.g., from about 0.014 to about 0.035, or about 0.014, or about 0.018, or about 0.035).

In some exemplary aspects, the assembly 20 that is at least partially insertable into the patient, at the at least one light emitting structure 30, can have a major transverse dimension 59 that is about 8 French (2.67 mm) or less (e.g., from about 5 French (1.67 mm) to about 8 French or from about 6 French (2 mm) to about 8 French). (“French” can be understood to be a measurement corresponding to thirds of a millimeter.) Accordingly, it is contemplated that the assembly 20 can be accommodated within an 8 French sheath or a 6 French sheath or a 5 French sheath. That is, the assembly 20 can have a maximum outer dimension (e.g., diameter) that accommodates a 6 French sheath or a 6 French sheath or a 5 French sheath.

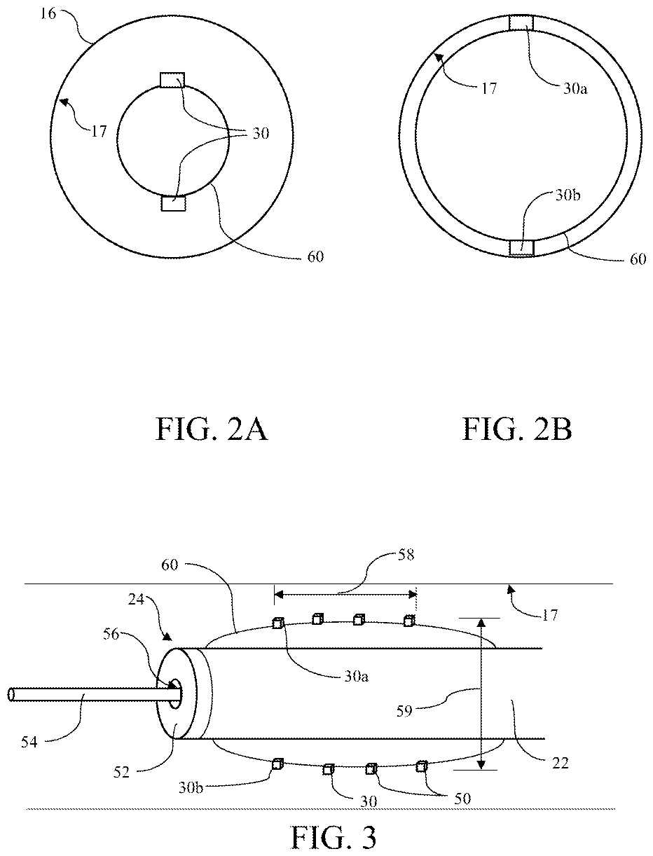

Referring to FIGS. 2A, 2B, and 3, in some aspects, the assembly 20 can comprise a bladder 60 that is inflatable. The light emitting structure(s) 30 can be coupled to the bladder 60 so that inflation of the bladder within the artery positions the light emitting structure(s) at or against the interior wall surface 17 of the artery 16.

In some aspects, the at least one light emitting structure 30 can comprise at least a first light emitting structure 30a and a second light emitting structure 30b that is offset from the first light emitting structure about a circumference of the bladder 60.

Referring to FIGS. 4-5, in some aspects, the insertional end portion 24 of the catheter 22 can comprise a resiliently elastic helical portion 64 that is configured to expand radially outwardly against the interior wall surface of the artery. The resiliently elastic helical portion 64 can have a central axis 66 and a radial spacing R from the central axis. In some aspects, the radial spacing can be from 1.5 millimeters to 10 millimeters when in a resting configuration (e.g., not compressed or stretched).

A sleeve 68 can be configured to receive the resiliently elastic helical portion 64 of the insertional end portion 24 of the catheter 22 in a compressed configuration. In use, the sleeve 68 (within which the resiliently elastic helical portion 64 is received) can be deployed within the subject or patient, and axial movement of the sleeve 68 (e.g., retraction) away from the resiliently elastic helical portion 64 of the insertional end portion of the catheter can permit radial expansion of the resiliently elastic helical portion.

In some optional aspects, the catheter 22 can comprise a resiliently elastic portion (e.g., optionally, the resiliently elastic helical portion 64) having an outer surface 70 that is configured to bias against the interior wall surface of the artery. The at least one light emitting structure 30 can be coupled to the outer surface of the resiliently elastic portion. In this way, the light emitting structure(s) 30 can contact the inner wall surface 17 of the artery 16.

In some aspects, the resiliently elastic helical portion 64 can be made from a polymer material (e.g., polyether block amide copolymer sold under the trademark PEBAX), a thermoplastic polyether urethane material (sold under the trademarks ELASTHANE or PELLETHANE), or other suitable materials having the desired properties, including a selected durometer. In some aspects, one or more additional materials may be added to the resiliently elastic helical portion 64 to help improve shape retention. In some aspects, for example, about 5 to 30 weight percent of siloxane can be blended with the resiliently elastic helical portion 64 (e.g., the thermoplastic polyether urethane material), and electron beam or gamma irradiation may be used to induce cross-linking of the materials. In other embodiments, the resiliently elastic helical portion 64 may be formed from different material(s) and/or have a different arrangement. In various optional aspects, the catheter 22 or a portion thereof (e.g., the resiliently elastic helical portion 64) can comprise a polymer material such as polyamide, polyimide, polyether block amide copolymer sold under the trademark PEBAX, polyethylene terephthalate (PET), polypropylene. aliphatic, polycarbonate-based thermoplastic polyurethane sold under the trademark CARBOTHANE, or a polyether ether ketone (PEEK) polymer that provides the desired flexibility.

The system 10 can further comprise at least one photosensitizer and an apparatus that is configured to deliver the at least one photosensitizer to the patient. For example. the apparatus that is configured to deliver the at least one photosensitizer to the patient can be an intravenous (IV) apparatus. In further aspects, the at least one photosensitizer can be administered orally. In some aspects, the photosensitizer can comprises tetracycline, demeclocycline, doxycycline, minocycline, or a combination thereof. In further aspects, other photosensitizers and antibiotics are contemplated.

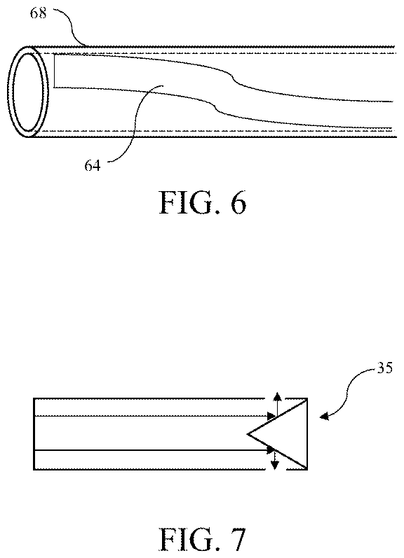

Referring to FIGS. 1-6, a method can comprise delivering at least one photosensitizer to a patient (e.g., orally or intravenously). The at least one light emitting structure 30 of the assembly 20 can be positioned within an artery of the patient. For example, angiographic guidance can be used to position the at least one light emitting structure of the assembly within the artery of the patient. The at least one light emitting structure of the assembly can then deliver light to an interior wall surface of the artery.

In embodiments in which the catheter 22 of the assembly 20 has a resiliently elastic portion, the resiliently elastic portion of the assembly can be biased against the interior wall surface 17 of the artery 16.

At least some of the light can have a wavelength from 300 nm to 600 nm.

In some aspects, the portion of the assembly can be inserted into a contralateral femoral artery or an ipsilateral tibial artery of the patient.

In embodiments in which the assembly 20 comprises a bladder 60 that is inflatable to position the light emitting structure(s) 30 at or against the interior wall surface 17 of the artery 16, the method can further comprise inflating the bladder to position the emitting structure(s) at or against the interior wall surface of the artery. In some optional aspects, the bladder 60 can be repeatedly deflated and inflated to permit movement of the at least one light emitting structure relative to the interior wall surface of the artery to permit positioning the at least one light emitting structure at different portions of the interior wall surface. For example, inflating and deflating the bladder can allow the catheter to rotate relative to the artery so that the position of the light emitting structures can change relative to the interior wall surface.

In embodiments in which the insertional end portion 24 of the catheter 22 comprises a resiliently elastic helical portion 64, the helical portion can be advanced or retracted within the artery to change the position of the light emitting structure(s).

The sleeve can retain the resiliently elastic helical portion 64 in a compressed configuration to permit insertion of the catheter and location of the resiliently elastic helical portion at the treatment site. The sleeve 68 can then be axially retracted from the resiliently elastic helical portion of the insertional end portion of the catheter to permit expansion of the resiliently elastic helical portion from a compressed configuration.

In some aspects, the photodynamic therapy disclosed herein can mitigate inflammation of an artery. In further aspects, the photodynamic therapy disclosed herein can inhibit, reduce, and/or stabilize plaque. The photodynamic therapy disclosed herein can be an adjunctive treatment. That is, the photodynamic therapy can be used in combination with another plaque treatment, such as angioplasty. For example, an angioplasty treatment can be performed to open a blocked artery, and the photodynamic therapy can subsequently be performed in the same location as the angioplasty. The photodynamic treatments can eliminate the cycle of inflammation. That is, plaque can be inhibited from re-growing where it has been treated.

As can be understood, systems for providing photodynamic therapy to a coronary artery have neither the sufficient length to reach the tibial artery, nor the sufficient rigidity to navigate the leg arteries, nor the transverse dimensions that can fit into a tibial artery.

By positioning the light source at or against the interior wall surface of the artery, blood flow need not be occluded (e.g., with an inflatable body) or flushed with saline, and the blood does not absorb or otherwise diminish intensity of the light against the interior wall surface of the artery. Accordingly, in some aspects, the system 10 does not comprise an inflatable body for occluding blood flow. Still further, by positioning the light source at or against the interior wall surface of the artery, a sufficient intensity of light can be achieved with a relatively lower luminosity light source as compared to a light source that is spaced from the interior wall surface and, therefore, emits more diffuse light across a greater surface area.

### Exemplary Aspects

In view of the described products. systems. and methods and variations thereof. herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A system comprising:
an assembly that is at least partially insertable into a patient, the assembly comprising:
   a catheter having an insertional end portion, wherein the catheter is configured to permit positioning of the insertional end portion at a treatment site of an artery having an interior wall surface: and
   at least one light emitting structure that is coupled to the insertional end portion of the catheter, wherein the assembly is configured to position the at least one light emitting structure at or against the interior wall surface of the artery.

Aspect 2: The system of aspect 1, wherein the assembly further comprises:
an optical fiber having an insertional first end portion and an opposed second end portion, the optical fiber comprising:
   a core: and
   a cladding circumferentially enclosing at least a portion of the core:
a light source that is configured to deliver light into the opposed second end portion of the optical fiber,
wherein the at least one light emitting structure comprises a portion of the cladding that is configured to permit light to pass therethrough.

Aspect 3: The system of aspect 2, wherein the light source is a laser.

Aspect 4: The system of aspect 2 or aspect 3, wherein the portion of the cladding that is configured to permit light to pass therethrough comprises at least one aperture.

Aspect 5: The system of any one of aspects 2-4, wherein the light source is configured to emit light at a wavelength from about 300 nm to about 600 nm.

Aspect 6: The system of aspect 1, wherein the at least one light emitting structure comprises a light emitting device.

Aspect 7: The system of aspect 6, wherein the light emitting device comprises an LED, a super luminescent diode, a laser diode, a light emitting polymer, or combinations thereof.

Aspect 8: The system of any one of aspects 6-7, wherein the light emitting device is configured to emit light at a wavelength from about 300 nm to about 600 nm.

Aspect 9: The system of any one of the preceding aspects, wherein the artery is a tibial artery.

Aspect 10: The system of any one of the preceding aspects, wherein the assembly that is at least partially insertable into the patient comprises a marker at a known position relative to the at least one light emitting structure.

Aspect 11: The system of any one of the preceding aspects, wherein the catheter has a length, wherein the at least one light emitting structure comprises a plurality of light emitting structures that are spaced along the length of the catheter.

Aspect 12: The system of any one of aspects 1-10, wherein the catheter has a length, wherein the at least one light emitting structure is elongate along the length of the catheter.

Aspect 13: The system of any one of the preceding aspects, further comprising a guidewire, wherein the catheter defines an inner lumen that is configured to receive the guidewire therein.

Aspect 14: The system of any one of the preceding aspects, wherein the guidewire is sufficiently rigid to position the at least one light emitting structure in an artery below a knee of the patient.

Aspect 15: The system of any one of the preceding aspects, wherein the guidewire has a diameter from about 0.014 inches to about 0.035 inches.

Aspect 16: The system of any one of the preceding aspects, wherein the assembly that is at least partially insertable into the patient, at the at least one light emitting structure, has a major transverse dimension that is about 8 French or less.

Aspect 17: The system of aspect 16, wherein the major transverse dimension of the assembly that is at least partially insertable into the patient, at the at least one light emitting structure, is from about 6 French to about 8 French.

Aspect 18: The system of any one of the preceding aspects, wherein the assembly that is at least partially insertable into the patient further comprises a bladder that is inflatable. wherein the at least one light emitting structure is coupled to the bladder so that inflation of the bladder within the artery is configured to position the at least one light emitting structure at or against the interior wall surface of the artery.

Aspect 19: The system of aspect 18, wherein the at least one light emitting structure comprises at least a first light emitting structure and a second light emitting structure that is offset from the first light emitting structure about a circumference of the bladder.

Aspect 20: The system of any one of aspects 1-17, wherein the insertional end portion of the catheter comprises a resiliently elastic helical portion that is configured to expand radially outwardly against the interior wall surface of the artery.

Aspect 21: The system of aspect 20, wherein the resiliently elastic helical portion has a central axis and a radial spacing from the central axis, wherein the radial spacing is from 1.5 millimeters to 10 millimeters.

Aspect 22: The system of aspect 20 or aspect 21, further comprising: a sleeve that is configured to receive the resiliently elastic helical portion of the insertional end portion of the catheter in a compressed configuration, wherein axial movement of the sleeve away from the resiliently elastic helical portion of the insertional end portion of the catheter permits radial expansion of the resiliently elastic helical portion.

Aspect 23: The system of any one of aspects 1-17, wherein the catheter comprises a resiliently elastic portion having an outer surface that is configured to bias against the interior wall surface of the artery, wherein the at least one light emitting structure is coupled to the outer surface of the resiliently elastic portion.

Aspect 24: The system of any one of the preceding aspects, wherein the at least one light emitting structure is configured to emit light along a treatment length from about 50 mm to about 200 mm.

Aspect 25: The system of any one of the preceding aspects, wherein the catheter has a working length from about 90 cm to about 120 cm.

Aspect 26: The system of any one of the preceding aspects, further comprising: at least one photosensitizer: and an apparatus that is configured to deliver the at least one photosensitizer to the patient.

Aspect 27: The system of any one of the preceding aspects, wherein the at least one photosensitizer comprises tetracycline, demeclocycline, doxycycline, minocycline, or a combination thereof.

Aspect 28: A method comprising:
delivering at least one photosensitizer to a patient:
positioning at least one light emitting structure of an assembly within an artery of the patient, wherein the assembly comprises:
   a catheter having an insertional end portion: and
   the at least one light emitting structure, wherein the at least one light emitting structure is coupled to the insertional end portion of the catheter; and
delivering, by the at least one light emitting structure of the assembly, light to an interior wall surface of the artery.

Aspect 29: The method of aspect 28, wherein the artery is a tibial artery.

Aspect 30: The method of aspect 28 or aspect 29, wherein the catheter of the assembly has a resiliently elastic portion, wherein positioning the at least one light emitting structure of the assembly within the artery of the patient comprises biasing the resiliently elastic portion of the assembly against the interior wall surface of the artery.

Aspect 31: The method of any one of aspects 28-30, wherein the positioning the at least one light emitting structure of the assembly within the artery of the patient comprises using angiographic guidance to position the at least one light emitting structure of the assembly within the artery of the patient.

Aspect 32: The method of any one of aspects 28-31, wherein at least a portion of the light has a wavelength from 300 nm to 600 nm.

Aspect 33: The method of any one of aspects 28-32, wherein the at least one photosensitizer comprises tetracycline, demeclocycline, doxycycline, minocycline, or a combination thereof.

Aspect 34: The method of any one of aspects 28-33. wherein the positioning the at least one light emitting structure of the assembly within the artery of the patient comprises inserting a portion of the assembly into a contralateral femoral artery or an ipsilateral tibial artery of the patient.

Aspect 35: The method of any one of aspects 28-34, wherein the assembly that is at least partially insertable into the patient further comprises a bladder that is inflatable, wherein the at least one light emitting structure is coupled to the bladder, the method further comprising inflating the bladder to position the at least one light emitting structure at or against the interior wall surface of the artery.

Aspect 36: The method of aspect 35, further comprising repeatedly deflating and inflating the bladder to permit movement of the at least one light emitting structure relative to the interior wall surface of the artery to permit positioning the at least one light emitting structure at different portions of the interior wall surface.

Aspect 37: The method of any one of aspects 28-34, wherein the insertional end portion of the catheter comprises a resiliently elastic helical portion that is configured expand radially outwardly against the interior wall surface of the artery.

Aspect 38: The method of aspect 37, further comprising retracting a sleeve axially from the resiliently elastic helical portion of the insertional end portion of the catheter to permit expansion of the resiliently elastic helical portion from a compressed configuration.

Although the foregoing invention has been described in some detail by way of illustration and example for purposes of clarity of understanding, certain changes and modifications may be practiced within the scope of the appended claims.

What is claimed is:

1. A system comprising:
an assembly that is at least partially insertable into a patient, the assembly comprising:
a catheter having an insertional end portion, wherein the catheter is configured to permit positioning of the insertional end portion at a treatment site of an artery having an interior wall surface: and
at least one light emitting structure that is coupled to the insertional end portion of the catheter, wherein the assembly is configured to position the at least one light emitting structure at or against the interior wall surface of the artery.

2. The system of claim 1, wherein the assembly further comprises:
an optical fiber having an insertional first end portion and an opposed second end portion, the optical fiber comprising:
a core: and
a cladding circumferentially enclosing at least a portion of the core:
a light source that is configured to deliver light into the opposed second end portion of the optical fiber,
wherein the at least one light emitting structure comprises a portion of the cladding that is configured to permit light to pass therethrough.

3. The system of claim 2, wherein the light source is a laser.

4. The system of claim 2, wherein the portion of the cladding that is configured to permit light to pass therethrough comprises at least one aperture.

5. The system of claim 2, wherein the light source is configured to emit light at a wavelength from about 300 nm to about 600 nm.

6. The system of claim 1, wherein the at least one light emitting structure comprises a light emitting device.

7. The system of claim 6, wherein the light emitting device comprises an LED, a super luminescent diode, a laser diode, a light emitting polymer, or combinations thereof.

8. The system of claim 6, wherein the light emitting device is configured to emit light at a wavelength from about 300 nm to about 600 nm.

9. The system of claim 1, wherein the artery is a tibial artery.

10. The system of claim 1, wherein the assembly that is at least partially insertable into the patient comprises a marker at a known position relative to the at least one light emitting structure.

11. The system of claim 1, wherein the catheter has a length, wherein the at least one light emitting structure comprises a plurality of light emitting structures that are spaced along the length of the catheter.

12. The system of claim 1, wherein the catheter has a length, wherein the at least one light emitting structure is elongate along the length of the catheter.

13. The system of claim 1, further comprising a guidewire, wherein the catheter defines an inner lumen that is configured to receive the guidewire therein.

14. The system of claim 1, wherein the guidewire is sufficiently rigid to position the at least one light emitting structure in an artery below a knee of the patient.

15. The system of claim 1, wherein the guidewire has a diameter from about 0.014 inches to about 0.035 inches.

16. The system of claim 1, wherein the assembly that is at least partially insertable into the patient, at the at least one light emitting structure, has a major transverse dimension that is about 8 French or less.

17. The system of claim 16, wherein the major transverse dimension of the assembly that is at least partially insertable into the patient, at the at least one light emitting structure, is from about 6 French to about 8 French.

18. The system of claim 1, wherein the assembly that is at least partially insertable into the patient further comprises a bladder that is inflatable, wherein the at least one light emitting structure is coupled to the bladder so that inflation of the bladder within the artery is configured to position the at least one light emitting structure at or against the interior wall surface of the artery.

19. The system of claim 18, wherein the at least one light emitting structure comprises at least a first light emitting structure and a second light emitting structure that is offset from the first light emitting structure about a circumference of the bladder.

20. The system of claim 1, wherein the insertional end portion of the catheter comprises a resiliently elastic helical portion that is configured to expand radially outwardly against the interior wall surface of the artery.

21. The system of claim 20, wherein the resiliently elastic helical portion has a central axis and a radial spacing from the central axis, wherein the radial spacing is from 1.5 millimeters to 10 millimeters.

22. The system claim 20, further comprising: a sleeve that is configured to receive the resiliently elastic helical portion of the insertional end portion of the catheter in a compressed configuration, wherein axial movement of the sleeve away from the resiliently elastic helical portion of the insertional end portion of the catheter permits radial expansion of the resiliently elastic helical portion.

23. The system of claim 1, wherein the catheter comprises a resiliently elastic portion having an outer surface that is configured to bias against the interior wall surface of the artery, wherein the at least one light emitting structure is coupled to the outer surface of the resiliently elastic portion.

24. The system of claim 1, wherein the at least one light emitting structure is configured to emit light along a treatment length from about 50 mm to about 200 mm.

25. The system of claim 1, wherein the catheter has a working length from about 90 cm to about 120 cm.

26. The system of claim 1, further comprising:
at least one photosensitizer; and
an apparatus that is configured to deliver the at least one photosensitizer to the patient.

27. The system of claim 1, wherein the at least one photosensitizer comprises tetracycline, demeclocycline, doxycycline, minocycline, or a combination thereof.

28. A method comprising:
delivering at least one photosensitizer to a patient;
positioning at least one light emitting structure of an assembly of the system of claim 1 within an artery of the patient; and
delivering, by the at least one light emitting structure of the assembly, light to an interior wall surface of the artery.

29. (canceled)

30. (canceled)

31. (canceled)

32. (canceled)

33. (canceled)

34. (canceled)

35. (canceled)

36. (canceled)

37. (canceled)

38. (canceled)
